(19) United States
(12) Patent Application Publication
(10) Pub. No.: US 2002/0115134 A1
(43) Pub. Date: Aug. 22, 2002

(54) Simplified production of bispecific antibody fragments

(76) Inventor: Gundram Jung, Germering (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/115,074

(22) Filed: Apr. 4, 2002

Related U.S. Application Data
(63) Continuation of application No. 09/230,392, filed on Jun. 8, 1999, now abandoned.

(51) Int. Cl. C12P 21/06; C07K 16/00

(52) U.S. Cl. 435/68.1; 530/388.1

(57) ABSTRACT

The invention relates to a method for producing bispecific proteins, preferably antibody fragments, characterized in that purification steps so far considered necessary can be dropped, thereby substantially simplifying the entire method and making it more cost-effective.

The invention relates to the production of bispecific antibody fragments for the immunotherapy of tumors in humans and animals.

The activation of the body's own immune system against malignant diseases has been an old therapeutic objective which, however, has not become a reality up to date. During the last few years, scientists succeeded in substantially improving the prerequisites required for attaining this aim. The key structures for activating the immune system on the surface of T cells have been identified and characterized. In essence, these key structures are the antigen-detecting T cell receptor/CD3 complex and the CD28 molecule as potentially most important co-stimulating receptor.

It is possible to construct bispecific antibodies such that they specifically bind with the one half to tumor cells and with the other half activate these central “molecular triggers” of the immune system. The activated cells are capable of effectively killing tumor cells (Jung and Müller-Eberhard, Immunol. Today 9 (1988), 257).

Meanwhile, the anti-tumor effect of bispecific antibodies has been shown in numerous in vitro test set-ups and also in several mouse-tumor models (survey in Beun et al., Immunol. Today 21 (1994), 2413).

In the first local applications in humans the peritoneal growth of ovarian carcinoma and glioblastoma could be favorably influenced (Nitta et al., Lancet 335 (1990), 368; Canevari et al., JNCI 87 (1995), 1463).

Therefore, there is reasonable expectation that bispecific antibodies will allow an effective immunotherapy of human tumors at least in certain clinical situations.

In order to avoid an unspecific activation of the immune cells via the Fc portion of the antibodies used, it is necessary to use bispecific antibody fragments lacking the Fc portion. In the prior art, Brennan et al. (Science 229 (1985), 81-83) reported a method for producing bispecific antibody fragments, wherein intact antibodies were fragmented by peptic digestion to F(ab′)₂ fragments, and the resulting F(ab′)₂ fragments were purified by column chromatography. Then, the disulfide bonds of the hinge region of the purified F(ab′)₂ molecule were digested by reduction in the presence of arsenite and the F(ab′)-SH fragments thus obtained were again purified by column chromatography, so as to then modify the reduced SH groups with the Eliman's reagent (DTNB) to F(ab′)-TNB. After further purification by column chromatography one of the two antibody fragments was reduced to F(ab′)-SH, purified by column chromatography and hybridized to the other F(ab′)-TNB fragment to give a bispecific F(ab′)₂ fragment. Finally, the bispecific antibody fragments thus obtained were purified by gel chromatography.

In the prior art, Jung et al. (Eur. J. Immunol. 21 (1991), 2431-2435) reported the modification of the above-mentioned method by simultaneously carrying out the digestion of the disulfide bonds in the hinge region of the F(ab′)₂ molecule and the blocking of the resulting SH groups with a protecting group so that one purification step of the method described by Brennan et al. could be dropped. All the same, the expense of time and money needed according to this method for the production of bispecific antibody fragments is so great that so far it has not been possible to produce the substance amounts that are required for a systemic application in humans and animals. Clinical testing of these promising reagents has been severely impeded by this fact. The development of a method that simplifies the production of bispecific antibody fragments even further would therefore allow to provide major amounts of such bispecific antibody fragments and their use in the immunotherapy of tumors.

The problem underlying the present invention was therefore to overcome the disadvantages of the prior art techniques described above and to provide a simplified method for producing bispecific antibody fragments.

The solution to the problem is achieved by the embodiments characterized in the claims.

It was surprisingly found that the reduction and modification of an antibody fragment obtained after peptic digestion can be carried out in one step without the purification step so far considered necessary in the prior art up to then. This finding allows a smaller number of process steps for the production of bispecific antibody fragments, the method additionally being characterized by a higher total yield of the desired end product. The method according to the invention is described for the production of bispecific F(ab′)₂ fragments from intact antibodies. Basically, this method can be used for the coupling of different proteins, as long as they contain disulfide bonds that can be reduced by TNB.

The invention thus relates to a method for producing bispecific proteins, preferably antibody fragments, characterized in that purification steps so far considered necessary can be dropped, thereby substantially simplifying the entire method and making it more cost-effective. In a preferred embodiment of the method of the invention the production of biologically active, bispecific antibody fragments essentially comprises the following steps:

(a) subjecting a first and a second antibody to a fragmentation step under conditions sufficient to cleave the Fc portion of the antibodies and to form F(ab′)₂ molecules;

(b) subjecting the respective products of step (a) without intermediate purification step to a reduction and modification step under conditions sufficient to enable digestion of the disulfide bonds in the hinge region of the F(ab′)₂ molecule and at the same time blocking the resulting SH groups with a protecting group;

(c) subjecting the respective products of step (b) to a purification step under conditions sufficient to remove the reaction adducts;

(d) subjecting the product of the first antibody of step (c) to a reduction step under conditions sufficient to cleave the protecting group of step (b);

(e) subjecting the product of step (d) to a purification step according to step (c); and

(f) subjecting the product of the second antibody of step (c) and the product of the first antibody of step (e) to a hybridization step under conditions sufficient to form bispecific F(ab′)₂ molecules.

The intact antibodies used in step (a) can be obtained according to methods known in the prior art (Current Protocols in Immunology, J. E. Codigan, A. M. Krvisbeck, D. H. Margulies, E. M. Shevack, W. Strober eds., John Wiley+Sons). It is also known from the art how to carry out the individual reaction and purification steps, and described, e.g., in Brennan et al. (Science 229 (1985), 81-83) and Jung et al. (Eur. J. Immunol. 21 (1991), 2491-2495).

In a preferred embodiment of the method according to the invention step (c) is carried out while simultaneously rebuffering the solution to pH 8.0, and/or step (e) while simultaneously rebuffering to pH 4.0.

In a likewise preferred embodiment of the method according to the invention the product of step (f) is subjected to a protein separation step under conditions resulting in the removal of non-hybridized F(ab′) molecules.

In another preferred embodiment the methods described above are carried out such that the antibody fragments obtained are sterile and/or pyrogen-free and/or essentially free of active viruses. This embodiment is suitable if the bispecific antibody fragments are intended for use in patients. This embodiment is, e.g., not necessarily required if the bispecific antibody fragments are intended for diagnostic purposes.

In a particularly preferred embodiment at least one of the antibodies is an antibody that recognizes surface antigens of T cells.

In another particularly preferred embodiment of the above-mentioned methods at least one of the antibodies is an antibody that recognizes surface antigens of tumor cells.

The method according to the invention allows to provide bispecific antibodies in sufficient amounts and at acceptable costs so that the therapy of tumors becomes possible for which the conventional methods have proven unsatisfactory. As a known example the lymphatically metastasizing malign melanoma is mentioned, for the successful treatment of which so far neither immunotherapeutic nor conventional methods of therapy have been available.

Thus, another preferred embodiment of the method according to the invention is characterized in that the antibody recognizing surface antigens of tumor cells is specific of melanomas.

In a particularly preferred embodiment of the above-described methods the antibody recognizing surface antigens of tumor cells is specific of CD3 or CD28.

In another particularly preferred embodiment the method according to the invention is characterized in that the fragmentation in step (a) comprises incubation for three hours at 37° C. of an antibody solution with pepsin and terminating the reaction by increasing the pH to 8.0 with Tris buffer.

In another embodiment of the method according to the invention the reduction and modification in step (b) comprises the addition of an equal volume of a DTNB-TNB mixture for 20 hours at room temperature.

In another preferred embodiment of the method according to the invention the purification step in steps (c) and (e) comprises gel filtration on Superdex 200 (c) and/or Sephadex G20 columns. The skilled person knows from the prior art how to choose the conditions for the purification of the products (c) and (e) using the above-mentioned columns. Also, the person skilled in the art may find other methods of purification of the products obtained by the method according to the invention from the prior art (Current Protocols in Immunology, J. E. Codigan, A. M. Krvisbeck, D. H. Margulies, E. M. Shevack, W. Strober eds., John Wiley+Sons).

In another preferred embodiment of the method according to the invention the reduction step in step (d) comprise the incubation of the product of step (c) in 0.1 mM DTT. This embodiment is particularly preferred and uses—as compared with the methods known in the art, see, e.g., Brennan et al. (Science 229 (1985), 81-83) and Jung et al. (Eur. J. Immunol. 21 (1991), 2491-2495)—very small, almost stoichiometric DTT concentrations with respect to the protein-SH groups blocked by TNB, which minimizes the probability of a reduction of the disulfide bonds between heavy and light chains. in this manner, it is not only the total yield of bispecific antibody fragments but also the quality of the products that can be increased by using the method according to the invention.

In another particularly preferred embodiment of the method the hybridization in step (f) is brought about by reaction of equal volumina of F(ab′)-TNB in 0.1 M phosphate buffer of pH 8.0 and F(ab′)-SH in 0.02 M acetate buffer of pH 4.0.

The figures show:

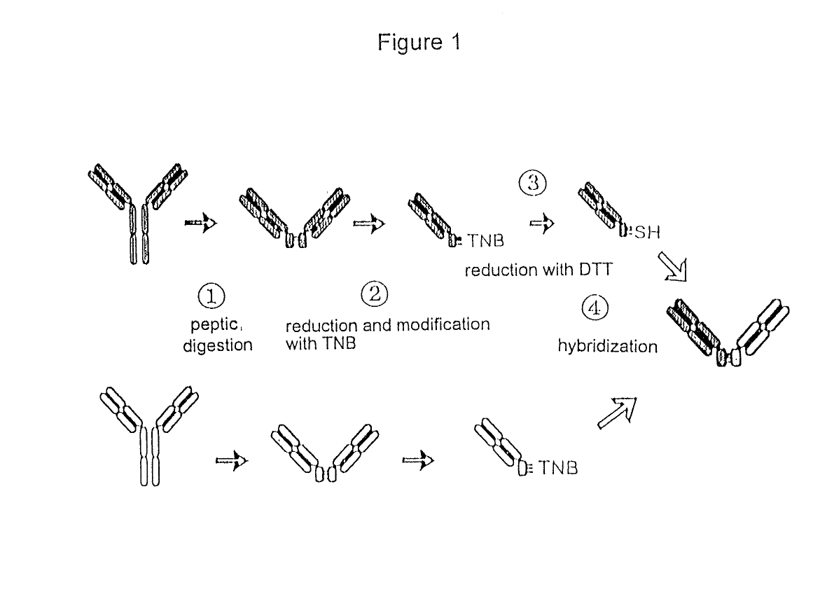

FIG. 1 Production of a bispecific F(ab′)₂ fragment according to the method of the invention.

The examples serve to illustrate the invention.

### EXAMPLE 1

Production of Bispecific F(ab′)₂ Antibody Fragments

The starting point of the method according to the invention are intact antibodies which are obtained as described in the prior art (Current Protocols in Immunology, J. E. Codigan, A. M. Krvisbeck, D. H. Margulies, E. M. Shevack, W. Strober eds., John Wiley+Sons). For this example, antibodies were used that are specific of a melanoma-associated proteoglycan or of the human CD3 molecule associated with the antigen-specific T cell receptor. These antibodies were treated with pepsin (Sigma) for three hours at 37° C. in acetate buffer of pH 4.0 so as to cleave off the Fc portion of the antibody (FIG. 1(1)). The reaction was terminated by increasing the pH value to 8 with Tris buffer and the resulting solution was incubated for 20 hours at room temperature with an equal volume of a mixture of 5,5′-dithiobis-2-nitrobenzoic acid (DTNB; Sigma) and thionitrobenzoate (TNB) (FIG. 1(2)). The molar ratio of the DTNB-TNB mixture is 20:30 and is adjusted by incubating for a few minutes a 40 mM DTNB solution with a 10 mM DTT solution. After repeated reduction of one of the two modified F(ab′) fragments with 0.1 mM DTT (Sigma) for one hour at 25° C. (FIG. 1(3)) the F(ab′)-TNB and F(ab′)-SH fragments thus obtained are mixed and hybridized for 1 hr at 25° C. to give a bispecific F(ab′)₂ fragment (FIG. 1(4)). The bispecific F(ab′)₂ fragments obtained were purified by gel filtration on a Superdex 200 column. The yield based on the amount of intact antibodies used was about 20%. As compared to the methods described in the prior art the yield could thus be increased by 50-100%.

### EXAMPLE 2

Functionality and Stability of the Bispecific F(ab′)₂ Fragments

All antibodies used and the bispecific constructs produced thereof were tested for their functionality and stability according to standard methods (Jung et al., Eur. J. Immunol. 21 (1991), 2431-2435). The functionality and stability of the F(ab′)₂ fragments produced according to the method of the invention was comparable to that of the fragments produced according to conventional methods. This refers, inter alia, to the binding to human tumor material, the activity in lymphocyte proliferation and cytotoxicity tests and the stability under in vivo conditions; incubated in human serum at 37° C. the constructs used were functionally stable for at least 6 days, in phosphate buffer at 5° C. for several months.

### EXAMPLE 3

Test of the Bispecific F(ab′)₂ Fragments in B16 Melanoma Cells of the Mouse

The bispecific antibody fragments produced according to the method of the invention were also tested for their in vivo activity in animal tests. According to these tests, the above-mentioned F(ab′)₂ fragments are capable of inhibiting the growth of melanoma cells in mice so that more than half of the animals treated survive whereas 100% of all untreated animals die.

1. Method for producing biologically active, bispecific antibody fragments, comprising the steps of:
(a) subjecting a first and a second antibody to a fragmentation step under conditions sufficient to cleave off the Fc portion of the antibodies and to form F(ab′)₂ molecules;
(b) subjecting the respective products of step (a) without intermediate purification step to a reduction and modification step under conditions sufficient to enable digestion of the disulfide bonds in the hinge region of the F(ab′)₂ molecule and at the same time blocking the resulting SH groups with a protecting group;
(c) subjecting the respective products of step (b) to a purification step under conditions sufficient to remove the reaction adducts;
(d) subjecting the product of the first antibody of step (c) to a reduction step under conditions sufficient to cleave the protecting group of step (b);
(e) subjecting the product of step (d) to a purification step according to step (c); and
(f) subjecting the product of the second antibody of step (c) and the product of the first antibody of step (e) to a hybridization step under conditions sufficient to form bispecific F(ab′)₂ molecules.

2. The method according to claim 1, characterized in that step (c) is carried out while simultaneously rebuffering the solution to pH 8.0, and/or step (e) while simultaneously rebuffering to pH 4.0.

3. The method according to claim 1 or 2, characterized by carrying out the following step:
subjecting the product of step (f) to a protein separation step under conditions resulting in the removal of non-hybridized F(ab′)₂^{•} molecules.
^{•} should actually read: “F(ab′)”

4. The method according to any one of claims 1 to 3, characterized in that the antibody fragments obtained are sterile, pyrogen-free and and essentially free of active viruses.

5. The method according to any one of claims 1 to 4, characterized in that at least one of the antibodies recognizes surface antigens of T cells.

6. The method according to any one of claims 1 to 5, characterized in that at least one of the antibodies recognizes surface antigens of tumor cells.

7. The method according to claim 6, characterized in that the antibody recognizing surface antigens of tumor cells is specific of melanomas.

8. The method according to any one of claims 5 to 7, characterized in that the antibody recognizing surface antigens of tumor cells is specific of CD3 or CD28.

9. The method according to any one of claims 1 to 8, characterized in that the fragmentation in step (a) comprises incubation for three hours at 37° C. of an antibody solution with pepsin and terminating the reaction by increasing the pH to 8.0 with Tris buffer.

10. The method according to any one of claims 1 to 9, characterized in that the reduction and modification in step (b) comprises the addition of an equal volume of a DTNB/TNB mixture for 20 hours at room temperature.

11. The method according to any one of claims 1 to 10, characterized in that the purification step in steps (c) and (e) comprises gel filtration on Superdex 200 (c) and/or Sephadex G20 columns.

12. The method according to any one of claims 1 to 11, characterized in that the reduction step in step (d) comprises the incubation of the product in 0.1 mM DTT.

13. The method according to any one of claims 1 to 12, characterized in that the hybridization in step (f) is brought about by reaction of equal volumina of Fab^{†}-TNB in 0.1 M phosphate buffer of pH 8.0 and Fab^{‡}-SH in 0.02 M acetate buffer of pH 4.0.
^{†} should actually read: “F(ab′)”
^{‡} should actually read: “F(ab′)”
